(19) United States
(12) Patent Application Publication
(10) Pub. No.: US 2008/0167621 A1
(43) Pub. Date: Jul. 10, 2008

(54) Multi-Barrel Syringe Having Integral Manifold

(76) Inventors: Gary S. Wagner, Independence, KY (US); Andrew D. Speth, St. Louis, MO (US)

Correspondence Address: Mallinckrodt Inc., 675 McDonnell Boulevard, HAZELWOOD, MO, 63042, US

(21) Appl. No.: 11/914,140

(22) PCT Filed: May 12, 2006
(86) PCT No.: PCT/US06/18495

§ 371 Date: Nov. 12, 2007

Related U.S. Application Data
(60) Provisional application No. 60/681,355, filed on May 16, 2005.

(51) Int. Cl. A61M 5/19; A61B 6/00

(52) U.S. Cl. 604/191; 600/432

(57) ABSTRACT

In certain embodiments, a system includes an integral manifold syringe. The integral manifold syringe may include a multi-barrel body having a first barrel and a second barrel, a manifold having first, second, third, and fourth ports, wherein the first and second ports are coupled to the first and second barrels, respectively. The integral manifold syringe also may include a flow control core disposed rotatably inside the manifold at a plurality of positions, wherein the flow control core has different flow pathway arrangements between the first, second, third, and fourth ports at the plurality of positions.

### FIELD OF THE INVENTION

The present invention relates generally to syringes and, more particularly to, a multi-fluid delivery system for medical fluids.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Nuclear medicine utilizes radioactive material for diagnostic and therapeutic purposes by injecting a patient with a small dose of the radioactive material, which concentrates in certain organs or biological regions of the patient. Radioactive materials typically used for nuclear medicine include Technetium-99m, Indium-113m, and Strontium-87m among others. Some radioactive materials naturally concentrate toward a particular tissue, for example, iodine concentrates toward the thyroid. However, radioactive materials are often combined with a tagging or organ-seeking agent, which targets the radioactive material for the desired organ or biologic region of the patient. These radioactive materials alone or in combination with a tagging agent are typically referred to as radiopharmaceuticals in the field of nuclear medicine. At relatively lower doses of the radiopharmaceutical, a radiation imaging system (e.g., a gamma camera) provides an image of the organ or biological region that collects the radiopharmaceutical. Irregularities in the image are often indicative of a pathologic condition, such as cancer. Higher doses of the radiopharmaceutical may be used to deliver a therapeutic dose of radiation directly to the pathologic tissue, such as cancer cells.

In certain applications, such as nuclear medicine, a plurality of fluids may be exchanged with a particular patient. Unfortunately, each fluid injection or withdrawal generally involves a separate syringe or other fluid exchange device. For example, each syringe or fluid exchange device may include a length of tubing coupled to the patient. Unfortunately, a significant amount of fluid is generally trapped inside the tubing and the particular syringe or device, thereby causing considerable fluid wastage. Furthermore, each separate syringe or fluid exchange device results in an incremental increase in the amount of trapped and wasted fluid. In addition, the plurality of separate syringes or fluid exchange devices unfortunately increases the potential for contamination due to the incrementally greater number of possible contamination sites. For example, each syringe or device may include a plurality of connection points, which can become contaminated during a particular injection procedure.

### SUMMARY

The present invention, in certain embodiments, is directed to fluid flow control between multiple barrels of an integral multi-barrel syringe. In context of the present application, the term integral or integrated refers to a single, unitary, one-piece unit or structure. In addition, the term integral or integrated may include a multi-piece structure that includes parts joined or incorporated together to form a substantially single-piece whole. For example, the multi-piece unitary structure may include parts assembled in direct contact or at least in close proximity with one another in a manner such that the parts are substantially in separable from each other. In the present application, the terms integral, integrated, united, or unitized may be used interchangeably, but are intended to include both a single, unitary, one-piece structure and a multi-piece unitary structure. In certain embodiments, one or more one-way valves (e.g., check valves) and/or multi-way valves (e.g., two-way, three-way, four-way, etc.) may be disposed between the multiple barrels and one or more ports. In addition, these valves may be disposed in a multi-passage structure or a multi-way manifold, which may be integral or united with the multi-barrel syringe. Together, the multi-barrel syringe, the multi-passage structure or multi-way manifold, and the valves may be a substantially single unitary structure or a multi-piece united structure, which may be described generally as an integral manifold syringe. The integral manifold syringe may substantially reduce the likelihood of fluid contamination, spillage, and general wastage between the syringe barrels and a patient and/or one or more external devices or containers. For example, the integral manifold syringe may eliminate a number of potential contamination sites and lengths of tubing.

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of features and aspects that may not be set forth below.

In accordance with a first aspect of the present invention, there is provided a system including an integral manifold syringe. The integral manifold syringe may include a multi-barrel body having a first barrel and a second barrel, a manifold having first, second, third, and fourth ports, wherein the first and second ports are coupled to the first and second barrels, respectively. The integral manifold syringe also may include a flow control core disposed rotatably inside the manifold at a plurality of positions, wherein the flow control core has different flow pathway arrangements between the first, second, third, and fourth ports at the plurality of positions.

In accordance with a second aspect of the present invention, there is provided a system including an integral multi-barrel syringe. The integral multi-barrel syringe may include a first plunger disposed in a first barrel, a second plunger disposed in a second barrel, and a multi-passage structure coupled to the first and second barrels, wherein the multi-passage structure may include first and second ports downstream of the first and second barrels. The integral multi-barrel syringe also may include a one-way valve disposed in the multi-passage structure and a multi-way valve disposed in the multi-passage structure, wherein the multi-way valve may include an actuator.

In accordance with a third aspect of the present invention, there is provided a method including changing fluid flow pathways in a multi-way manifold between a plurality of barrels of a unitized multi-barrel syringe and a plurality of ports.

Various refinements exist of the features noted above in relation to the various aspects of the present invention. Further features may also be incorporated in these various aspects as well. These refinements and additional features may exist individually or in any combination. For instance, various features discussed below in relation to one or more of the illustrated embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE FIGURES

These and other features, aspects, and advantages of the present invention will become better understood when the following detailed description is read with reference to the accompanying figures in which like characters represent like parts throughout the figures, wherein:

FIG. 1 is a schematic of an embodiment of a multi-barrel delivery system with a check valve proximate an outlet of one of the barrels;

FIG. 2 is a perspective view of an embodiment of a unitized multi-barrel delivery system similar to that of FIG. 1;

FIG. 3 is a schematic of another embodiment of a multi-barrel delivery system with a first check valve proximate an outlet of a first barrel and a second check valve proximate an outlet of a second barrel;

FIG. 4 is a perspective view of an embodiment of a unitized multi-barrel delivery system similar to that of FIG. 2;

FIG. 5 is a partially exploded, perspective view of another embodiment of a multi-barrel delivery system including two syringes and a front end assembly;

FIG. 6A is a schematic of an embodiment of a multi-barrel delivery system having a three- way valve open to an outlet of a barrel and a delivery assembly;

FIG. 6B is an enlarged schematic of an embodiment of the three-way valve of FIG. 6A with the valve open to outlets of two barrels;

FIG. 6C is an enlarged schematic of an embodiment of the three-way valve of FIG. 6A with the valve open to an outlet of a barrel and the delivery assembly;

FIG. 7A is a schematic of another embodiment of a multi-barrel delivery system having a source of saline and a three-way valve in fluid communication with barrel outlets, a delivery assembly, and a check valve associated with one of the barrel outlets;

FIG. 7B is an enlarged schematic of an embodiment of the three-way valve of FIG. 7A with the valve open to the source of saline solution and an outlet of a first syringe barrel;

FIG. 7C is an enlarged schematic of an embodiment of the three-way valve of FIG. 7A with the valve open to the source of saline solution and the delivery assembly;

FIG. 8A is a schematic of another embodiment of a multi-barrel delivery system having a source of saline solution connected proximate an outlet associated with a barrel, a two-way valve, and two check valves;

FIG. 8B is an exploded view of an embodiment of the two-way valve in the closed position;

FIG. 9 is a perspective view of an embodiment of a multi-barrel delivery system similar to that of FIG. 8A;

FIG. 10A is a schematic of another embodiment of a multi-barrel delivery system having a source of medical fluid connected proximate an outlet of one of the barrels, a two-way valve, and two check valves;

FIG. 10B is an exploded view of an embodiment of the two-way valve in the closed position;

FIG. 11 is a perspective view of an embodiment of an integral manifold syringe having a multi-barrel syringe and an integral stopcock manifold or valve-controlled manifold;

FIG. 12 is a top view of an embodiment of the integral manifold syringe as illustrated in FIG. 11;

FIG. 13 is a perspective view of an embodiment of the integral stopcock manifold or valve- controlled manifold as illustrated in FIGS. 11 and 12;

FIG. 14 is a cross-sectional bottom view of an embodiment of the integral stopcock manifold or valve-controlled manifold as illustrated in FIG. 13, further illustrating a no flow configuration between first, second, third, and fourth ports;

FIG. 15 is a cross-sectional bottom view of an embodiment of the integral stopcock manifold or valve-controlled manifold as illustrated in FIG. 13, further illustrating a first syringe fluid exchange configuration having the first port fluidly coupled to the third port;

FIG. 16 is a cross-sectional bottom view of an embodiment of the integral stopcock manifold or valve-controlled manifold as illustrated in FIG. 13, further illustrating a second syringe fluid exchange configuration having the second port fluidly coupled to the third port;

FIG. 17 is a cross-sectional bottom view of an embodiment of the integral stopcock manifold or valve-controlled manifold as illustrated in FIG. 13, further illustrating a dual syringe fluid exchange configuration having both the first and second ports fluidly coupled to the third port;

FIG. 18 is a cross-sectional bottom view of an embodiment of the integral stopcock manifold or the valve-controlled manifold as illustrated in FIG. 13, further illustrating a vent configuration or supplemental fluid exchange configuration having the third port fluidly coupled to the fourth port;

FIG. 19 is a top view of an embodiment of the integral manifold syringe as illustrated in FIGS. 11 and 12, further illustrating a simultaneous multi-syringe injection configuration having a syringe link or coupling between first and second syringes;

FIG. 20 is a flowchart illustrating an embodiment of a nuclear medicine process utilizing one or more of the embodiments illustrated in FIGS. 1-19;

FIG. 21 is a block diagram illustrating an embodiment of a radiopharmacy or radiopharmaceutical production system utilizing one or more of the embodiments illustrated in FIGS. 1-19; and

FIG. 22 is a block diagram illustrating an embodiment of a nuclear imaging system utilizing one or more of the embodiments illustrated in FIGS. 1-19.

### DETAILED DESCRIPTION OF SPECIFIC EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Again, in context of the present application, the term integral or integrated may include a single unitary structure or one-piece unit. In addition, the term integral or integrated may include a multi-piece united structure, which includes parts joined or incorporated together to form the whole. For example, the multi-piece unitary structure may include parts assembled in direct contact or at least in close proximity with one another. In the present application, the terms integral, integrated, united, or unitized may be used interchangeably, but are intended to include both a single unitary structure and a multi-piece united structure.

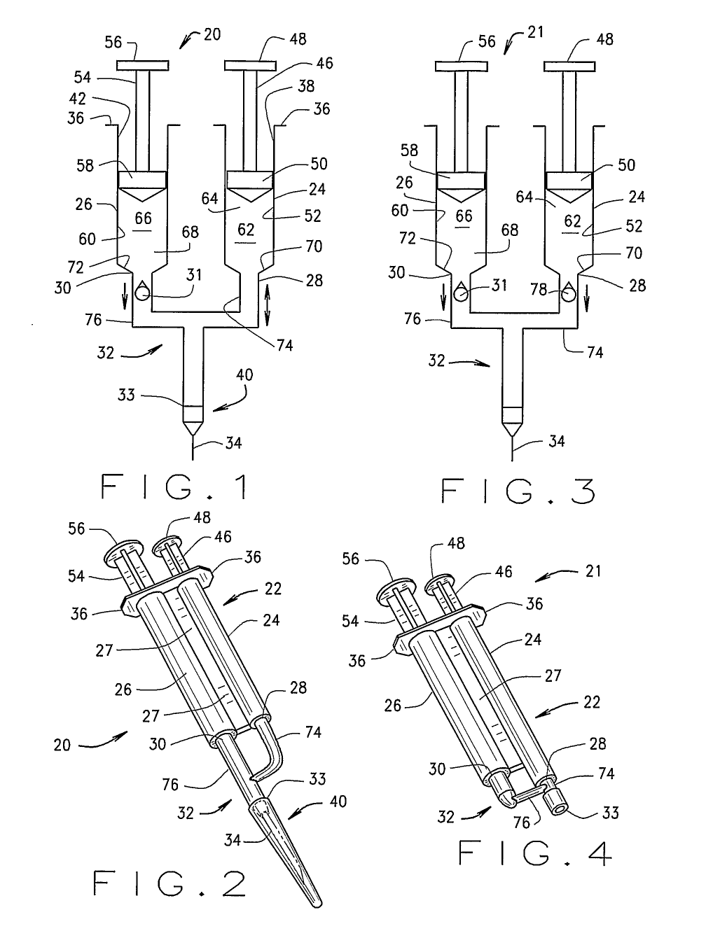

Referring now to FIGS. 1 and 2, FIG. I is a schematic of a two barrel delivery system 20 and FIG. 2 is a perspective view of one embodiment of the two barrel delivery system of FIG. 1. The following detailed description and corresponding illustrations of exemplary embodiments generally refer to multi-barrel delivery systems that include two syringe-type barrels. However, the scope of various embodiments includes multi-barrel delivery systems including more than two (e.g., three, four, five, six, seven, eight, or even more) syringe-type barrels. The integrated or unitized two barrel delivery system 20 may include an integral or unitized body 22, which may include a first barrel 24 and a second barrel 26 connected via an appropriate joint 27. While this joint 27 is illustrated as being integral with the first and second barrels 24, 26 of the deliver system 20, other embodiments of the deliver system 20 may include other appropriate manners of interconnecting the first and second barrels 24, 26. For example, the first and second barrels 24, 26 may be coupled together via an epoxy, glue, or another adhesive, or the first and second barrels 24, 26 may be coupled together via one or more clamps, straps, latches, snap-fit mechanisms, or other fasteners, or a combination of adhesives and/or fasteners.

As discussed in detail below, a variety of flow control features may be incorporated into the two barrel delivery system 20. The first barrel may have an outlet 28, and the second barrel may have a separate outlet 30. The unitized two barrel delivery system 20 may have at least one check valve 31 (or the like) proximate the outlet 30 of the second barrel 26. Other embodiments of the delivery system 31 may include other appropriate locations for the check valve(s) 31. While no check valve is associated with the first barrel 24 of the delivery system 20, other embodiments of the delivery system 20 may include one or more check valves (or the like) associated with the first barrel 24. The unitized two barrel delivery system 20, and, specifically, the outlets 28, 30, may be in fluid communication with a front end assembly 32. In certain embodiments, the front end assembly 32 may embody a multi-way manifold or multi-passage structure, which may house one or more one-way valves (e.g., check valves) and/or one or more multi-way valves (e.g., two-way, three-way, or four-way valves). The front end assembly 32 may include a fitting 33 for connecting to a patient delivery assembly 40 (e.g., a needle 34). The patient delivery assembly 40 may refer to any appropriate delivery assembly capable of at least assisting in enabling the conveyance of medical fluid to a patient. For instance, the patient delivery assembly of some embodiments may refer to tubing and/or a needle-free connection (e.g., having a conventional luer fitting). A unitized finger grip 36 may be defined at an open end 38 of the first barrel 24 and at an open end 42 of the second barrel 26. Some embodiments of the delivery system 20 may or may not include the finger grip 36. Other embodiments may include an alterative design and/or location for the finger grip 36.

A first pushrod 46 may have an integral thumb tab 48 on one end and a plunger 50 (sometimes referred to as a proximal plunger) on the other end. The plunger 50 may form a seal with an inside wall 52 of the first barrel 24. A second pushrod 54 may have an integral thumb tab 56 on one end and a plunger 58 (sometimes referred to as a proximal plunger) on the other end. The plunger 58 may form a seal with an inside wall 60 of the second barrel 26. In certain embodiments, the first pushrod 46 may slide back and forth along the inside wall 52 and/or the second pushrod 54 may slide back and forth along the inside wall 60 in response to imposing at least some minimum threshold amount of pressure on the respective thumb tabs 48 and/or 56. In other words, the first and second pushrods 46, 54 may be “slidably positioned” in the first and second barrels 24, 26. It should be noted that some embodiments may not include elongate pushrods connected to respective plungers. For instance, some embodiments (e.g., designed for use with power injectors) may include a plunger without an associated elongate pushrod. Further, the pushrods 46, 54 may be generally utilized to bias or move the plungers 50, 58. Accordingly, any of a wide range of sizes, shapes and designs of pushrods may be appropriate depending on, for example, the desired use of the delivery system.

The plunger 50 and the inside wall 52 of the first barrel 24 may define a first chamber 62 for containing a first medical fluid 64. The plunger 58 and the inside wall 60 of the second barrel 26 may define a second chamber 66 for containing a second medical fluid 68. The first medical fluid 64 may be any medical fluid appropriate for administration to a patient. Further, the second medical fluid may be the same as or different from the first medical fluid and may be any medical fluid appropriate for administration to a patient. For instance, in some embodiments, the first medical fluid may be a radiopharmaceutical or a contrast agent and/or the second medical fluid may be a biocompatible flush (e.g., heparin solution, sterilized water, glucose solution, saline, or another suitable substance).

At the end of the first barrel 24, opposite the open end 38, is a terminus 70 of the barrel 24. Associated with the terminus 70 is the outlet 28 previously mentioned. At the end of the second barrel 26, opposite the open end 42, is a terminus 72 of the barrel 26. The terminus 70 and the terminus 72 may be referred to as a “conical end” as shown, or they may be other shapes, contours, and/or designs. A first flow conduit 74 may be in fluid communication with the outlet 28 of the first barrel 24 on one end of the first flow conduit 74, and the patient delivery assembly 40 (here, needle 34) may be in fluid communication with another other end of the conduit 74. A second flow conduit 76 may be in fluid communication with the outlet 30 of the second barrel 26 on one end and the patient delivery assembly 40 on the other end, which as previously noted may be a needle 34 in this figure.

The first flow conduit 74 may be bidirectional, as indicated by the flow arrows in FIG. 1. In other words, the first flow conduit 74 may be designed to enable medical fluid to be both drawn into and discharged from the barrel 24 (e.g., in response to movement of the pushrod 46). The check valve 31 in the second flow conduit 76 may substantially block or entirely prevent bidirectional flow in the second flow conduit 76 in certain embodiments. This check valve 31 may at least generally inhibit fluid in the front end assembly 32 from backflowing into the second chamber 66. Accordingly, the second flow conduit 76 may be characterized, in at least one regard, as effectively allowing only unidirectional flow, as indicated by the flow arrow in FIG. 1. As such, fluid can only be discharged from (as opposed to drawn into) the chamber 66, through outlet 30 and the flow conduit 76 and past the check valve 31. A luer fitting 33 or other appropriate interconnection device may be formed on or attached to the front end assembly 32 for engagement with the patient delivery assembly 40 which, as noted, is not limited to the needle 34.

To discharge the medical fluid 64 from the chamber 62 of the first barrel 24, pressure may be applied to the thumb tab 48 of the pushrod 46 causing the plunger 50 to slide down the inside wall 52 of the first barrel 24. To draw fluid into the chamber 62 of the first barrel 24, the thumb tab 48 may be withdrawn from the barrel 24 causing the plunger 50 to slide away from the terminus, creating negative pressure in the chamber 62 to draw a fluid therein. In some applications, it may be desirable to check for vein patency, e.g., whether or not the fluid delivery assembly is in fluid communication with the blood in a patient's vein. When checking for vein patency, it may be desirable to draw blood into the chamber 62, or at least into the conduit 74, assuming that one or both may be transparent or at least generally translucent. Once vein patency has been confirmed, the first medical fluid may then be administered to a patient. Checking for vein patency may be optional, depending on the nature of the administration site in the patient and other factors.

To discharge the second medical fluid 68 from the chamber 66 of the second barrel 26, pressure may be applied to the thumb tab 56 of the pushrod 54 causing the plunger 58 to slide down the inside wall 60 of the barrel 26. As the plunger 58 slides down the barrel, the second medical fluid may pass through the outlet 30, through the front end assembly 32 (including through the check valve 31), and out the patient delivery assembly 40 to the patient. The check valve 31 may substantially block or entirely prevent fluid from being drawn into the second barrel 26. The front end assembly 32, as shown in FIGS. 1 and 2, may include a first flow conduit 74, a second flow conduit 76, a first check valve 31, and a fitting 33. The fitting 33 may be utilized to interconnect the patient delivery assembly 40 which, as noted, can take any number of different forms. However, in the illustrated embodiment, the patient delivery assembly 40 includes the needle 34 with the front end assembly 32. Some embodiments of the delivery system 20 may not include a fitting 33 and may allow for direct connection of the patient delivery assembly 40 to the front end assembly 32. In other embodiments, the patient delivery assembly 40 may be integral with the front end assembly 32, substantially alleviating a need to utilize a fitting.

Referring now to FIGS. 3 and 4, the unitized two barrel delivery system 21 in FIG. 3 may be generally similar to that of FIG. 1, except for the addition of a second check valve 78. Further, the unitized two barrel delivery system 21 of FIG. 4 may be generally similar to that of FIG. 2, except the front end assembly 32 exhibits a different configuration. Accordingly, generally corresponding components in FIGS. 3 and 4 will be referred to using the same identification numbers as FIGS. 1 and 2.

Still referring to FIGS. 3 and 4, the second check valve 78 may be positioned in the first flow conduit 74 to substantially block or entirely prevent fluid flow into the first chamber 62. This second check valve 78 may be utilized to generally inhibit fluid backflow into the first chamber 62. Unlike the unitized two barrel delivery system 20 of FIGS. 1 and 2, the delivery system 21 shown in FIGS. 3 and 4 may generally isolate both of the medical fluids 64, 68 in the respective first and second chambers 62, 66 relative to external contamination. For example, in contrast to the embodiments of FIGS. 1 and 2, the first medical fluid 64 in the embodiments of FIGS. 3 and 4 may beneficially be prevented from being contaminated with any other fluid due to the presence of the second check valve 78 in the first flow conduit 74. The unitized two barrel delivery system 21 of FIG. 4 does not (although other embodiments may) have a needle 34 attached to the fitting 33. The first flow conduit 74 and the second flow conduit 76 may be shaped differently than FIG. 2, but the fluid flow paths may be substantially similar. The flow conduit 74 may be in fluid communication with the outlet 28 of the first barrel 24 on one end and with the fitting 33 on the other end. The fitting 33 may be in fluid communication with a patient delivery assembly.

In the embodiments of FIGS. 3 and 4, the second check valve 78 may substantially block or entirely prevent reverse flow into the first chamber 62. The flow conduit 76 may be in fluid communication with the outlet 30 of the second barrel 26 on one end and with the fitting 33 on the other end. The fitting 33 may be in fluid communication with a patient delivery assembly. The first check valve 31 may substantially block or entirely prevent reverse flow into the second chamber 66. The front end assembly 32, as shown in FIG. 4, may include a first flow conduit 74, a second flow conduit 76, a first check valve 31, a second check valve 78, and a fitting 33. The fitting 33 may enable interconnection of the front end assembly 32 with the patient delivery assembly.

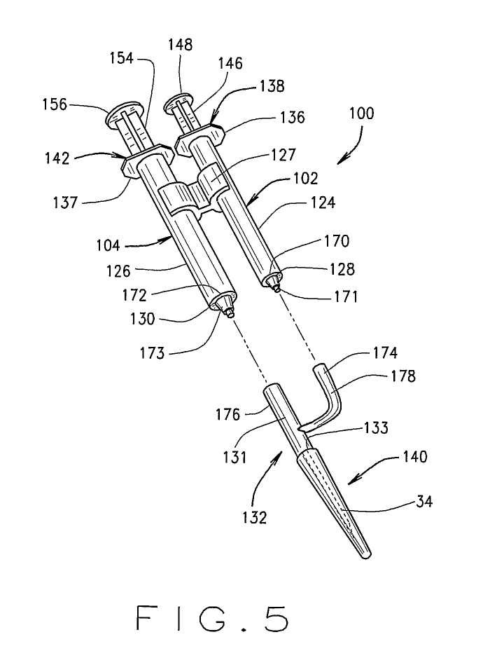

FIG. 5 is a perspective view of a delivery system 100 that may include a first syringe 102 and a second syringe 104, both of which may be removably connected to a front end assembly 132 of the delivery system 100. In certain embodiments, the front end assembly 132 may embody a multi-way manifold or multi-passage structure, which may house one or more one-way valves (e.g., check valves) and/or one or more multi-way valves (e.g., two-way, three-way, or four-way valves). These syringes 102, 104 are shown in this figure disconnected from the front end assembly 132. The flow paths and some of the components of the delivery system 100 in FIG. 5 may be similar to the unitized two barrel delivery system 20, shown in FIG. 1. The components in FIG. 5 which are common with components in FIG. 1 will be given the corresponding identification number as that of FIG. 1, except the common components will include a prefixial “1”. For example, the thumb tab 48 in the system 20 of FIG. 1 at least generally corresponds with a thumb tab 148 of the delivery system 100 of FIG. 5.

In the illustrated embodiment of FIG. 5, the first syringe 102 may define a first barrel 124 and the second syringe 104 may define a second barrel 126, wherein the first barrel 124 may be connected to the second barrel 126 by a clamp 127. The first and second syringes 102, 104 may be custom made or may be purchased off the shelf from a variety of different suppliers for use in the delivery system 100. The clamp 127 can be designed in different sizes to accommodate syringes of different sizes. Further, the configuration of the clamp 127 is not limited to the design shown in this figure. Any clamp or other mechanism for holding a plurality of syringes together (e.g., even one or more rubber bands and/or tape) may be appropriate for some embodiments. In certain embodiments, the clamp 127 may include one or more adhesives, threaded fasteners, latches, or other securing features. However, the illustrated clamp 127 may have a pair of opposite C-shaped structures that generally snap-fit about the exterior surfaces of the first and second barrels 124, 126.

The first barrel 124 may have an outlet 128 and the second barrel 126 may have a separate outlet 130 in fluid communication with the front end assembly 132 while connected to the two syringes 102, 104. A check valve 131 may be positioned in the front end assembly 132 proximate the outlet 130, when the syringes 102,104 are connected to the front end assembly 132. A fitting 133 may be positioned on the end of the front end assembly 132 opposite the syringes 102, 104. The fitting 133 may enable the front end assembly 132 to be interconnected to a patient delivery assembly 140, which in this figure may be a needle 134 drawn in phantom. However, the patient delivery assembly 140 could be characterized by any of several embodiments, as previously discussed. A first finger grip 136 may be defined at an open end 138 of the first syringe 102 and a second finger grip 137 may be defined at a second open end 142.

A first pushrod 146 may have an integral thumb tab 148 on one end and a first plunger (sometimes referred to as a proximal plunger) on the other end. The first plunger may form a seal with an inside wall of the first syringe 102. A second pushrod 154 may have an integral thumb tab 156 on one end of and a second plunger (sometimes referred to as a proximal plunger) on the other end. The second plunger may form a seal with an inside wall of the second syringe 104. In certain embodiments, the first pushrod 146 and the second pushrod 154 may generally slide back and forth along the inside walls of the first and second syringes 102, 104 in response to pressure from the respective thumb tabs 148 and 156. In other words, the first and second pushrods 146, 154 may be “slidably positioned” in the barrels. As previously noted, some embodiments may not include pushrods connected to respective plungers. For instance, some embodiments for use with power injectors may include a plunger without an associated elongate pushrod. Further, the pushrods 146 and 154 may be generally utilized to bias or move the plungers. Accordingly, any of a wide range of sizes, shapes and designs of pushrods may be appropriate depending on the desired use of the syringe.

The first plunger 150 and the inside wall 152 of the first syringe 102 may define a first chamber 162, which may be a mechanism for containing a first medical fluid 164. The second plunger 158 and the inside wall 160 of the second syringe 104 may define a second chamber 166, which may be a mechanism for containing a second medical fluid 164. The plungers 150, 158 and inside walls 152, 160 may be similar to those shown in FIG. 1, and have been assigned the same identification numerals, preceded by the number “1”. The previous discussion concerning the first and second medical fluids 64, 68 may be applicable to the medical fluids 164 and 168 in this embodiment as well.

At the end of the first syringe 102, opposite the open end 138, is a terminus 170 of the barrel 124. The terminus 170 forms the outlet 128, as previously mentioned. A fitting 171 may be positioned at the outlet 128. At the end of the second syringe 104, opposite the open end 142, is a terminus 172 of the barrel 126. A fitting 173 may be positioned at the outlet 130. The terminus 170 and 172 may be similar to the design shown in FIG. 1. The terminus 170, 172 of these syringes 102, 104 may be referred to as a “conical end,” or they may be other shapes well known to those skilled in the art. When the two syringes are connected to the front end assembly 132, the first flow conduit 174 may be in fluid communication with the outlet 128 of the first syringe 102 on one end and the patient delivery assembly 140 on the other end. In the present embodiment, the patient delivery assembly 140 may include a needle 34 as shown in phantom. A second flow conduit 176 may be in fluid communication with the outlet 130 of the second syringe 104 on one end and the patient delivery assembly 40 on the other end, which as previously noted may be a needle 34.

The first flow conduit 174 may be bidirectional as discussed in connection with FIG. 1. In other words, the first flow conduit 174 may be designed to enable medical fluid to be both drawn into and discharged from the first syringe 102 (e.g., in response to movement of the pushrod 46). The check valve 131 in the second flow conduit may substantially block or entirely prevent bidirectional flow in the second flow conduit in certain embodiments. This check valve 131 may function to substantially block or entirely prevent fluid flow into the second chamber 166.

To discharge the medical fluid 164 from the chamber 162 of the barrel 124 of the first syringe 102, pressure may be applied to the thumb tab 148 of the pushrod 146 causing the plunger 150 to slide down the inside wall 152 of the barrel. To draw fluid into the chamber 162 of the first barrel 124, the thumb tab 148 may be withdrawn from the barrel 124 causing the plunger 150 to slide away from the terminus, creating negative pressure in the chamber 162 to draw a fluid therein. In some applications, it may be desirable to check for vein patency, e.g., whether or not the fluid delivery assembly is in fluid communication with the blood in a patient's vein. When checking for vein patency, it may be desirable to draw the blood into the chamber 162, or at least into the conduit 174, if it is translucent. Once vein patency has been confirmed, the first medical fluid may then be administered to a patient. Checking for vein patency may be optional, depending on the nature of the administration site in the patient and other factors.

To discharge the second medical fluid 168 from the chamber 166 of the second barrel 126 of the second syringe 104, pressure may be applied to the thumb tab 156 of the pushrod 154 causing the plunger 158 to slide down the inside wall 160 of the barrel 126. As the plunger 158 slides down the barrel, the second medical fluid may pass through the outlet 130, through the conduit 172, and out the patient delivery assembly 140 to the patient. The check valve 131 may substantially block or entirely prevent fluid from being drawn into the second barrel 126 in certain embodiments. The front end assembly 132 as shown in this figure may include a first flow conduit 174, a second flow conduit 176, a first check valve 131 and a fitting 133. The fitting 133 may connect to a patient delivery assembly 140, which as noted can take any number of different forms. In the present embodiment, the delivery assembly 140 may include the needle 134.

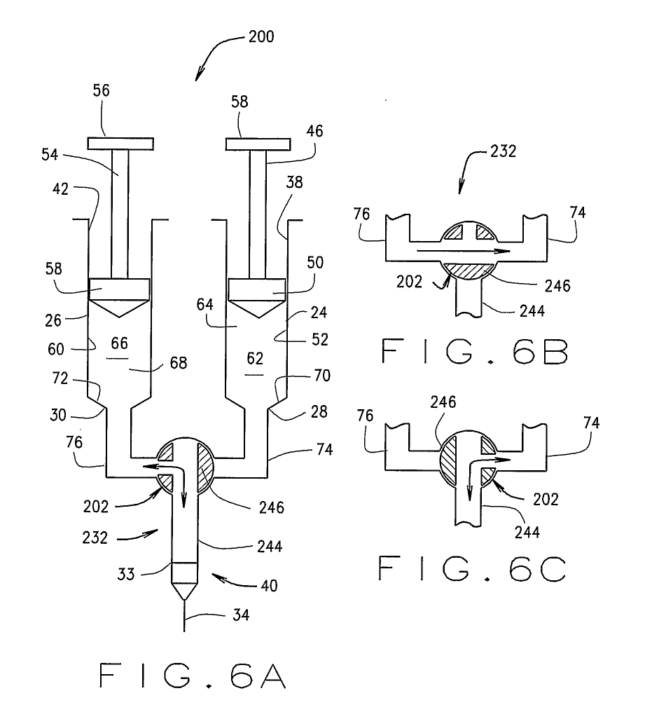

FIG. 6A is a schematic of a multi-barrel delivery system 300 having a three-way access valve 202. The barrels of the delivery system 200 can be formed in a unitized design (e.g., similar to FIGS. 2 and 4), or the barrels of the delivery system 200 can be components of two interconnected syringes (e.g., similar to the design of FIG. 5). Some of the flow paths and some of the components of the delivery system 200 in FIG. 6 may be substantially similar to that of the delivery system 20 shown in FIG. 1 and will be identified by common identification numbers. Those components with common numbers function in a similar manner which will not be repeated for the sake of brevity. The front end assembly 232 may include the access valve 202, the first flow conduit 74, the second flow conduit 76, a third flow conduit 244 (i.e., outlet conduit), and a fitting 33. In certain embodiments, the front end assembly 232 may embody a multi-way manifold or multi-passage structure, which may house one or more one-way valves (e.g., check valves) and/or one or more multi-way valves (e.g., two-way, three-way, or four-way valves). In the illustrated embodiment, the access valve 202 may be a three-way valve as discussed below.

The access valve 202 may be said to at least assist in the selective control of fluid flow between the first flow conduit 74, the second flow conduit 76, and the outlet conduit 244. A T-shaped valve element 246 may be rotated by the user to control fluid flow. A control unit for the three-way valve 202 can include a manual handle connected to the valve element 246 and/or electronic actuators. The first flow conduit 74 may be in fluid communication with the outlet 28 of the barrel 24 on one end and with the access valve 202 on the other end. The second flow conduit 76 may be in fluid communication with the outlet 30 of the second barrel 26 on one end and the access valve 202 on the other end. The outlet conduit 244 may be in fluid communication with the access valve 202 on one end and the fitting 33 on the other end. The outlet conduit 244 many enable fluid to leave the access valve 202 and pass through the patient delivery assembly 40 for administration of a medical fluid to the patient.

The delivery system 200 may be operated in the following manner. The access valve 202 may be selectively adjusted to the flow position shown in FIG. 6A allowing fluid to pass through the third flow conduit 244, the T-shaped valve element 246, and the second conduit 76. In the illustrated position of FIG. 6A, a second medical fluid 68 may be drawn into the chamber 66 of the second barrel 26. However, in the illustrated position of FIG. 6A, the T-shaped valve element 246 may substantially block or entirely prevent fluid flow between the third flow conduit 244 and the first conduit 74 and the corresponding chamber 62. Alternatively, the access valve 202 may be selectively adjusted to the flow position shown in FIG. 6C allowing fluid to pass through the third flow conduit 244, the T-shaped valve element 246, and the first flow conduit 74. In the illustrated position of FIG. 6C, a first medical fluid 64 may be drawn into the chamber 62 of the first barrel 24. However, in the illustrated position of FIG. 6C, the T-shaped valve element 246 may substantially block or entirely prevent fluid flow between the third flow conduit 244 and the second conduit 76 and the corresponding chamber 66. For purposes of this example, the first medical fluid 64 may be a radiopharmaceutical or a contrast agent, and the second medical fluid 68 may be an appropriate biocompatible flush (e.g., heparin solution, sterilized water, glucose solution, saline, etc.) although as previously discussed, the disclosed embodiments are not limited to these two fluids. The T-shaped valve element may be selectively adjusted back to the position shown in FIG. 6A.

The patient delivery assembly 40 may be connected to the vasculature of a patient (e.g., via a hypodermic needle inserted into a vein). The connection may be checked for patency of venous access from the second barrel 26. The T-shaped valve element 246 may be adjusted to the position shown in FIG. 6C, and at least some of the first medical fluid may be administered to the patient. The T-shaped valve element may be adjusted to the position in FIG. 6A, and at least a portion of the second medical fluid may be administered to the patient. Thus, the illustrated system 200 may enable sequential injection of multiple fluids, e.g., 64, 68, into a patient from the chambers 62, 66. The T-shaped valve element 246 may be selectively adjusted to the position of FIG. 6B, allowing fluid communication between the first conduit 74 and the second conduit 76. The remaining second medical fluid may be drawn/pushed from the chamber 66 of the second barrel 26 into the chamber 62 of the first barrel 24. The T-shaped valve element 246 may be selectively adjusted back to the position of FIG. 6C, and the residual first fluid and the remaining second fluid may be administered to the patient from the chamber 66 of the first barrel 24. The delivery system 200 may then be disposed of, using proper procedures (e.g., depending on the types of medical fluids administered).

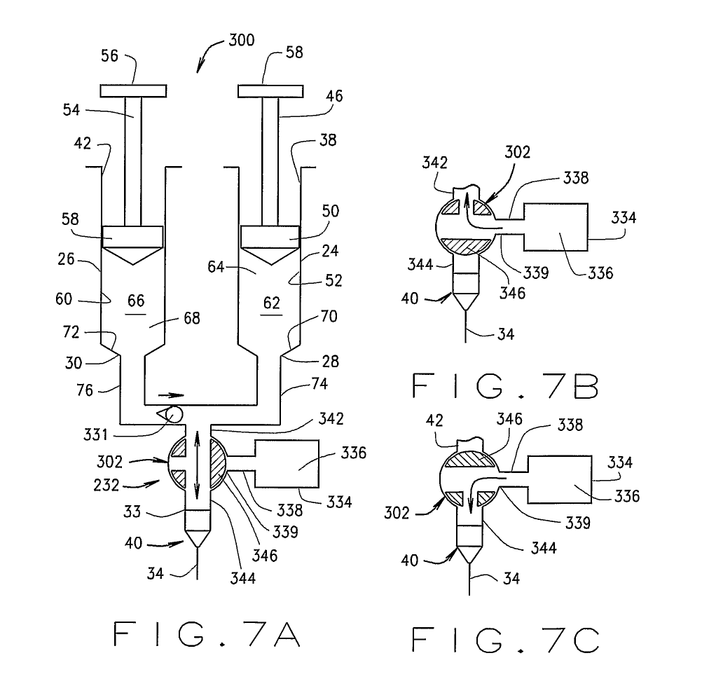

FIG. 7A is a schematic of another multi-barrel delivery system 300 having a three-way access valve 302 and a check valve 331. In this delivery system 300, the access valve 302 may be connected to a source 334 (e.g., a generally pliable or flexible bag) including a third medical fluid 336, which may sometimes be saline solution and/or other biocompatible flush. The barrels of the delivery system 300 can be formed in a unitized design (e.g., similar to FIGS. 2 and 4), or the barrels of the delivery system 300 may be components of two distinct, yet interconnected, syringes (e.g., similar to the design of FIG. 5). Some of the flow paths and some of the components of the delivery system 300 in FIG. 7A may be substantially similar to that of the delivery system 20 shown in FIG. 1 and will be identified by common identification numbers. Those components with common numbers function in an at least similar manner which will not be repeated for the sake of brevity. Tubing 338 or another type of conduit (e.g., flexible conduit) may connect the source 334 of the third medical fluid to the access valve 302. An outlet conduit 344 may connect the access valve 302 with the patient delivery device 40. An inlet conduit 342 may connect the first flow conduit 74 and the second flow conduit 76 with the access valve 302. The inlet conduit 342 may deliver fluid to the access valve 302 from either of the chambers 62 in the first barrel 24 or 66 in the second barrel 26. The outlet conduit 344 may convey fluid away from the access valve 302 and such fluid may come from the first chamber 62, the second chamber 66, or the third source 334 of the third medical fluid 336. The front end assembly 332 of this embodiment may include the access valve 302, the first flow conduit 74, the second flow conduit 76, the inlet conduit 342, the outlet conduit 344, a connector 339 for the tubing 338, and a fitting 33. In certain embodiments, the front end assembly 332 may embody a multi-way manifold or multi-passage structure, which may house one or more one-way valves (e.g., check valves) and/or one or more multi- way valves (e.g., two-way, three-way, or four-way valves).

The access valve 302 may be said to at least assist in the control of fluid flow between the first flow conduit 74, the second flow conduit 76, the tubing 338 and the outlet conduit 344. A T-shaped valve element 346 may be selectively rotated by the user to control fluid flow. A control unit for the access valve 302 can include a manual handle connected to the valve element 346 and/or an electronic actuator. The first flow conduit 74 may be in fluid communication with the outlet 28 of the barrel 24 on one end and with the inlet conduit 342 to the access valve 302 on the other end. The second flow conduit 76 may be in fluid communication with the outlet 30 of the second barrel 26 on one end and the inlet conduit 342 to the access valve 302 on the other end. The outlet conduit 344 may be in fluid communication with the three-way the access valve 202 on one end and the fitting 33 on the other end. The outlet conduit 344 may enable fluid to leave the access valve 302 and pass through the patient delivery assembly 40 for administration of a medical fluid to the patient.

The delivery system 300 may be operated in the following manner. The check valve 331 may substantially block or entirely prevent the medical fluid from being drawn through the access valve 302 and into the second chamber 66. Therefore, the second medical fluid may be prefilled before delivery to a user, or the user may remove the pushrod 54 from the open end 42 of the second barrel 26 to fill the chamber 66 of the second barrel. The access valve 302 may be selectively adjusted to the flow position shown in FIG. 7A, allowing fluid to pass through the outlet conduit 344, the T-shaped valve element 346, and the first flow conduit 74. In the illustrated position of FIG. 7A, a first medical fluid 64 may be drawn into the chamber 62 of the first barrel 24. In the alternative, the delivery system 300 may come prefilled with both a first and a second medical fluid.

For purposes of this example, the first medical fluid 64 may be a radiopharmaceutical or a contrast agent and the second medical fluid 68 may be a saline solution, although as previously discussed, the disclosed embodiments are not limited to these two fluids. The patient delivery assembly 40 may be connected to the vein of the patient. The connection may be checked for patency of venous access. The T-shaped valve element 346 may be selectively adjusted to the position shown in FIG. 7A and the first medical fluid may be administered to the patient. The T-shaped valve element 346 may be selectively adjusted or left in the position in FIG. 7A and a portion of the second medical fluid may be administered to the patient. The T-shaped valve element 346 may be selectively adjusted back to the position of FIG. 7B allowing fluid communication between the first conduit 74 and the source 334 of the third medical fluid 336. An aliquot of third medical fluid 336 may be drawn into the chamber 62 of the first barrel 24. The T-shaped valve element 346 may then be adjusted back to the position of FIG. 7A and the residual first fluid and the aliquot of the third medical fluid may be administered to the patient from the chamber 66 of the first barrel 24. The T-shaped valve element 346 may then be selectively adjusted to the position of FIG. 7C. The patient may then receive a drip of the third medical fluid from the source 334. When the drip is complete, the two barrel delivery system 300 with three-way valve 302 and check valve 331 may then be disposed, using proper procedures, depending on the types of medical fluids administered.

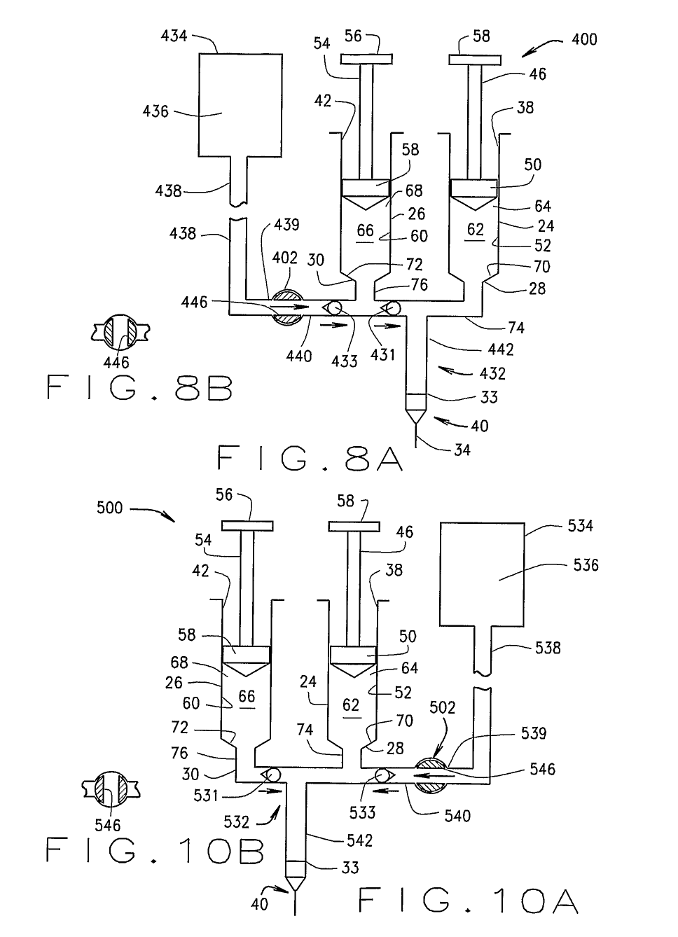

FIG. 8A is a schematic of another multi-barrel delivery system 400 having a two-way access valve 402, a first check valve 431, and a second check valve 433. In this delivery system 400, the access valve 402 may be connected to a source 434, such as a flexible bag or the like, of a third medical fluid 436. In certain embodiments, the medical fluid 436 may be a saline solution. The various barrels of the delivery system 400 can be formed in a unitized design (e.g., similar to FIGS. 2 and 4), or the barrels of the delivery system 400 can be components of two independent, interconnected, syringes (e.g., similar to the design of FIG. 5). Some of the flow paths and some of the components of the delivery system 400 in FIG. 8A may be substantially similar to that of the delivery system 20 shown in FIG. 1 and will be identified by common identification numbers. Those components with common numbers function in an at least generally similar manner which will not be repeated for the sake of brevity. Tubing 438 or another type of flexible conduit may connect the source 434 of the third medical fluid to the access valve 402. An outlet conduit 440 may connect the access valve 402 with the second flow conduit 76. A central conduit 442 may connect the first flow conduit 74 and the outlet conduit 440 with the patient delivery assembly 40. The tubing 438 may engage a connector 439 at the two-way valve 402 and may deliver the third medical fluid to the access valve 402. The outlet conduit 440 may take fluid away from the two-way valve 402 while open as shown in FIG. 8A. The front end assembly 432 of the delivery system 400 may include the access valve 402, the first check valve 431, the second check valve 433, the first flow conduit 74, the second flow conduit 76, the outlet conduit 440, the central conduit 442, a connector 439, and a fitting 33. In certain embodiments, the front end assembly 432 may embody a multi-way manifold or multi-passage structure, which may house one or more one-way valves (e.g., check valves) and/or one or more multi-way valves (e.g., two-way, three-way, or four-way valves).

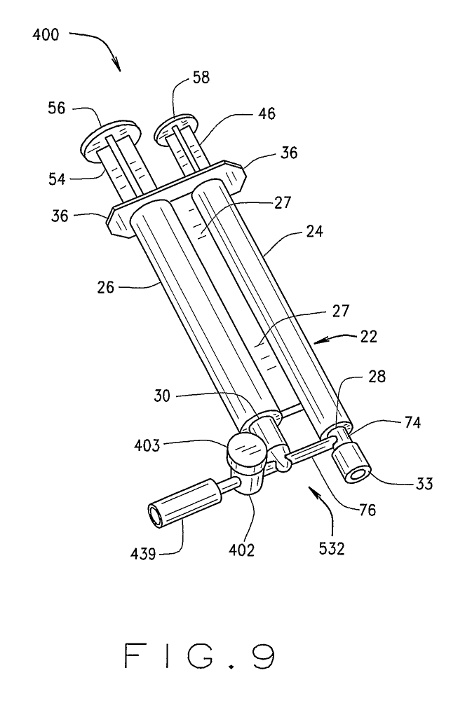

The access valve 402 may be utilized to assist in control of fluid flow between the first source of a third medical fluid 434 and the central conduit 442. A valve element 446 may be selectively adjusted by the user by rotation of the valve handle 403, better seen in FIG. 9, to control fluid flow. The first flow conduit 74 may be in fluid communication with the outlet 28 of the barrel 24 on one end and with the central conduit 442 on the other end. The second flow conduit 76 may be in fluid communication with the outlet 30 of the second barrel 26 on one end and the outlet conduit 440 on the other end. The outlet conduit 440 may be in fluid communication with the access valve 402 on one end and the central conduit 442 on the other end. The outlet conduit 440 may enable fluid to leave the access valve 402 and pass through the patient delivery assembly 40 for administration of a medical fluid to the patient.

The delivery system 400 may be operated in the following manner. The check valve 431 may substantially block or entirely prevent the second medical fluid from being drawn into the second chamber 66. Therefore, the second medical fluid may be prefilled before delivery to a user, or the user may remove the pushrod 54 from the open end 42 of the second barrel 26 to fill the chamber 66 of the second barrel. The access valve 402 may be selectively adjusted to the flow position shown in FIG. 8A allowing fluid to pass through the central conduit to the first flow conduit 74. This allows a first medical fluid 64 to be drawn into the chamber 62 of the first barrel 24. In the alternative, the two barrel delivery system 400 may come prefilled with both a first and a second medical fluid.

For purposes of this example, the first medical fluid 64 may be a radiopharmaceutical or a contrast agent, the second medical fluid 68 may be a saline solution, and the third medical fluid 436 may be a drip of saline solution, although as previously discussed, the disclosed embodiments are not limited to these fluids. The valve element 446 may be selectively adjusted to the closed position as shown in FIG. 8B. The patient delivery assembly 40 may be connected to the vein of the patient. In addition, the connection may be checked for patency of venous access by drawing blood into the first chamber 62 of the first barrel 24.

The first medical fluid may be administered to the patient from the first chamber 62 of the first barrel 24. A portion of second medical fluid 68 may be administered to the patient from the second chamber 66 of the second barrel 26. Furthermore, the remaining second medical fluid 68 can be drawn into the first chamber 62 from the second chamber 66. The residual first fluid and the aliquot of the second medical fluid may be administered to the patient from the chamber 62. In addition, the valve element 446 may be moved from the closed position of FIG. 8B to the open position of FIG. 8A allowing a drip of the third medical fluid 436 from the source 434. When the drip is complete, the two barrel delivery system 400 with two-way valve 402 and two check valves 431, 433 may then be disposed of using proper procedures, depending on the types of medical fluids administered. As can be seen from FIG. 8A, the first check valve 431 may substantially block or entirely prevent fluid flow into the chamber 66 of the second barrel 26 and the second check valve 433 may substantially block or entirely prevent fluid flow into the access valve 402.

FIG. 9 is a perspective view of a representative embodiment of the delivery system 400 described herein in relation to FIG. 8A, having a two-way access valve 402, a first check valve 431 (see FIGS. 8A and 8B), and a second check valve 433 (see FIGS. 8A and 8B). Some of the flow paths and some of the components of the delivery system 400 in FIG. 9 may be the same as the delivery system 21 shown in FIG. 4 and will be identified by common identification numbers. Those components with common numbers function in an at least generally similar manner which will not be repeated for the sake of brevity. The access valve 402 may include a handle 403 for adjusting the position of the valve element 446, as illustrated in FIGS. 8A, 8B, and 9. A connector 439 may connect to the access valve 402 and to the tubing 438 which runs to a source 434 of a third medical fluid 436, as illustrated in FIGS. 8A, 8B, and 9.

FIG. 1A is a schematic of another embodiment of a multi-barrel delivery system 500 having a two-way valve access 502, a first check valve 531, a second check valve 533, and a source 535 of a third medical fluid 536 in a different position than the embodiment 400 of FIG. 8A. In this delivery system 500, the access valve 502 may be located proximate the outlet 28 of the first barrel 24. The access valve 502 may be connected to a source 534, such as a flexible bag, of a third medical fluid 536. In certain embodiments, the third medical fluid 536 may be a saline solution. Some of the flow paths and some of the components of the delivery system 500 in FIG. 10A may be substantially similar to that of the delivery system 20 shown in FIG. 1 and will be identified by common identification numbers. Those components with common numbers function in an at least generally similar manner which will not be repeated for the sake of brevity. A connector 539 may connect the tubing 538 to the source 534 of the third medical fluid on one end and to a connector 539 on the access valve 502 on the other end. An outlet conduit 540 may connect the access valve 502 with the first flow conduit 74. A central conduit 542 may connect the first flow conduit 74 and the outlet conduit 540 with the patient delivery assembly 40. The outlet conduit 540 may take fluid away from the two-way valve 502 (while open as shown in FIG. 10A) and the first flow conduit 74. The front end assembly 532 of this embodiment may include the access valve 502, a first check valve 533, a second check valve 531, the first flow conduit 74, the second flow conduit 76, the outlet conduit 540, the central conduit 542, a connector 539, and a fitting 33. In certain embodiments, the front end assembly 532 may embody a multi-way manifold or multi-passage structure, which may house one or more one-way valves (e.g., check valves) and/or one or more multi-way valves (e.g., two-way, three-way, or four-way valves).

The access valve 502 may at least generally assist in the control of fluid flow between the third medical fluid 534 and the central conduit 542. A valve element 546 may be selectively adjusted by the user by rotating the valve element 546 to control fluid flow. The control unit for the access valve 502 can include manual handles and/or electronic controls associated with and/or connected to the valve element 546. The first flow conduit 74 may be in fluid communication with the outlet 28 of the barrel 24 on one end and with the central conduit 542 on the other end. The second flow conduit 76 may be in fluid communication with the outlet 30 of the second barrel 26 on one end and the central conduit 542 on the other end. The outlet conduit 540 may be in fluid communication with the access valve 502 on one end and the central conduit 542 on the other end. The outlet conduit 540 may enable fluid to pass through the access valve 502 and the patient delivery assembly 40 for administration of a third medical fluid to the patient.

The two barrel delivery system 500 may be operated in the following manner. The check valve 531 may substantially block or entirely prevent the second medical fluid from being drawn into the second chamber 66. Therefore, the second medical fluid may be prefilled before delivery to a user, or the user may remove the pushrod 54 from the open end 42 of the second barrel 26 to fill the chamber 66 of the second barrel. The access valve 502 may be selectively adjusted to the flow position shown in FIG. 10A allowing fluid to pass through the central conduit to the first flow conduit 74. This allows a first medical fluid 64 to be drawn into the chamber 62 of the first barrel 24. In the alternative, the two barrel delivery system 500 may come prefilled with both a first and a second medical fluid.

For purposes of this example, the first medical fluid 64 may be a radiopharmaceutical or a contrast agent, the second medical fluid 68 may be a saline solution, and the third medical fluid 536 may be a drip of saline solution, although as previously discussed, the disclosed embodiments are not limited to these fluids. The valve element 546 may be selectively adjusted to the closed position as shown in FIG. 10B. The patient delivery assembly 40 may be connected to the vein of the patient. In addition, the connection may be checked for patency of venous access by drawing blood into the first chamber 62 of the first barrel 24.

The first medical fluid may be administered to the patient from the first chamber 62 of the first barrel 24. A portion of second medical fluid 68 may be administered to the patient from the second chamber 66 of the second barrel 26. In addition, the remaining second medical fluid 68 may be drawn into the first chamber 62 from the second chamber 66. The residual first fluid and the aliquot of the second medical fluid may be administered to the patient from the chamber 62.

Furthermore, the valve element 546 may be moved from the closed position of FIG. 10B to the open position of FIG. 10A allowing fluid flow (e.g., a drip) of the third medical fluid 536 from the source 534 to the patient. When the fluid flow is complete, the delivery system 500 may then be disposed of, using proper procedures (e.g., depending on the types of medical fluids administered). As can be seen from FIG. 10A, the first check valve 531 may substantially block, entirely prevent, or at least generally inhibit fluid flow into the chamber 66 of the second barrel 26, and the second check valve 533 may substantially block, entirely prevent, or at least generally inhibit backflow of fluid to the access valve 502.

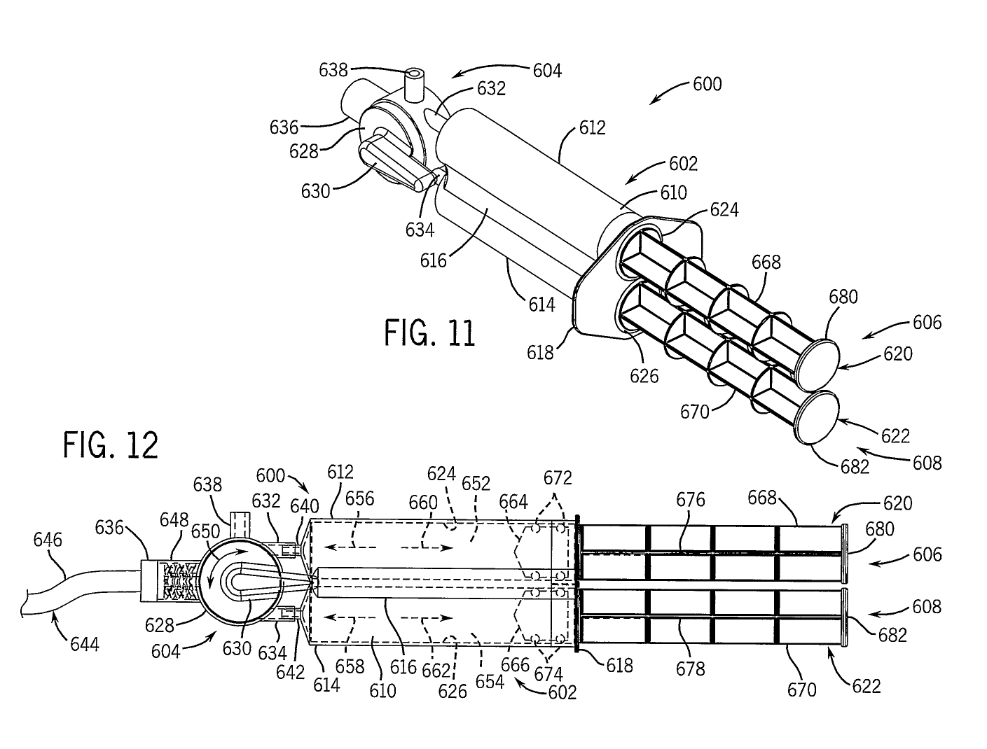

FIG. 11 is a perspective view of an exemplary embodiment of an integral manifold multi-fluid exchange system or integral manifold syringe 600 having a multi-barrel syringe 602 and an integral stopcock manifold or valve-controlled manifold 604. Again, as mentioned above, the term integral may include a single unitary structure or one-piece unit or, alternatively, a multi-piece united structure. As discussed in further detail below, the integral manifold syringe 600 may substantially reduce the likelihood of fluid contamination, spillage, and general wastage between the multi-barrel syringe 602 and a patient and/or one or more external devices or containers. For example, the integral stopcock manifold or valve-controlled manifold 604 may be integrally formed or fixably coupled to the multi-barrel syringe 602, thereby eliminating a number of potential contamination sites and lengths of tubing. The multi-barrel syringe 602 may include a first syringe 606 and a second syringe 608, which may be integrally coupled together via one or more lengths or joints. For example, the multi-barrel syringe 602 may include a unitized multi-barrel body 610 having a first barrel or cylindrical enclosure 612 and a second barrel or cylindrical enclosure 614. These first and second barrels or cylindrical enclosures 612, 614 may be integrally formed or coupled together via an intermediate lengthwise joint 616 and a unitized finger grip or flange 618. For example, a substantial portion or all of the unitized multi-barrel body 610 may be formed by molding or extruding the first and second barrels or cylindrical enclosures 612, 614 as a single unit or structure. Alternatively, the first and second barrels or cylindrical enclosures 612, 614 may be molded or extruded separately and then coupled together via a suitable adhesive or bonding material along the intermediate lengthwise joint 616. In addition, the unitized finger grip or flange 618 may be molded, extruded, or generally manufactured independently or integrally with the first and second barrels or cylindrical enclosures 612, 614.

The first and second syringes 606, 608 also may include first and second plungers 620, 622 disposed moveably within the first and second barrels or cylindrical enclosures 612, 614, respectively. More specifically, the first and second plungers 620, 622 may extend lengthwise or in generally parallel linear directions inwardly and outwardly from first and second chambers or cylindrical passages 624, 626 of the first and second barrels or cylindrical enclosures 612, 614 respectively. As discussed in further detail below, the first and second syringes 606, 608 may be operated sequentially or simultaneously to inject, extract, or generally exchange one or more fluids with a patient or an external device via the integral stopcock manifold or valve-controlled manifold 604. For example, the integral stopcock manifold or valve-controlled manifold 604 may enable fluid injection, extraction, or general exchange with the first syringe 606 completely independent from the second syringe 608, or vice versa. Alternatively, the integral stopcock manifold or valve-controlled manifold 604 may enable simultaneous fluid injection, extraction, or general exchange with both the first and second syringes 606, 608 and one or more external devices, containers, patients, and so forth. Moreover, the integral stopcock manifold or valve-controlled manifold 604 may enable a plurality of the integral manifold syringes 600 to be coupled in series, or in parallel, or a combination thereof. In this manner, the integral manifold syringe 600 can be adapted to a variety of multi-fluid injection, extraction, or general exchange scenarios in a medical environment or other application.

FIG. 12 is a top view of an embodiment of the integral manifold syringe 600 as illustrated in FIG. 11, further illustrating features of the multi-barrel syringe 602 and the integral stopcock manifold or valve-controlled manifold 604. As illustrated, the integral stopcock manifold or valve-controlled manifold 604 may include a stopcock manifold body or housing 628, a valve lever or manifold flow control actuator 630, and a plurality of ports. The stopcock manifold body or housing 628 may be described as a multi-way manifold or a multi-passage structure configured to support one or more valves, such as one-way valves (e.g., check valves), multi-way valves (e.g., two-way, three-way, four-way, etc.), electronic valves, manual valves, or a combination thereof. In the illustrated embodiment, the actuator 630 may be a hand-operable or manual control member. In alternative embodiments, the actuator 630 may include an electronic actuator, a motorized actuator, or a remote-controlled actuator, or a computer-controlled actuator, or a combination thereof. The integral stopcock manifold or valve-controlled manifold 604 may include a set of three, four, five, six, seven, eight, nine, ten, or more ports. In the illustrated embodiment, the integral stopcock manifold or valve-controlled manifold 604 includes a first port 632, a second port 634, a third port 636, and a fourth port 638. Other embodiments of the integral stopcock manifold or valve-controlled manifold 604 may include additional ports for each additional plunger, or additional outputs, or additional inputs to facilitate the injection, extraction, or exchange of fluids.

For example, the first and second ports 632, 634 may be coupled to first and second tip fittings or conduits 640, 642 extending from the first and second barrels or cylindrical enclosures 612, 614, respectively. The first and second ports 632, 634 may be integrally formed or coupled together with the first and second tip fittings or conduits 640, 642 via molding, adhesives, welding, or other suitable techniques. In other embodiments, the first and second ports 632, 634 and the first and second tip fittings or conduits 640, 642 may include male and female couplings or fittings, which can be removably secured together to create the integral manifold syringe 600. For example, the first and second ports 632, 634 and the first and second tip fittings or conduits 640, 642 may include male and female luer fittings, male and female threads, latches or snap fittings, compression fittings, or combinations thereof. For example, if threaded fittings or luer fittings are used to connect the first and second ports 632, 634 with the corresponding first and second tip fittings or conduits 640, 642, then the intermediate lengthwise joint 616 and the flange 618 may enable the first and second barrels or cylindrical enclosures 612, 614 to temporarily rotate during the connection process. Subsequently, the intermediate lengthwise joint 616 and/or the flange 618 may secure or fix the first and second barrels or cylindrical enclosures 612, 614, thereby securing the connection between the first and second ports 632, 634 and the corresponding first and second tip fittings or conduits 640, 642. Again, if one or more additional syringes are integrated with the multi-barrel syringe 602, then the integral manifold syringe 600 may include additional ports and connections with the integral stopcock manifold or valve-controlled manifold 604.

In addition to the first and second ports 632, 634, the third port 636 may be coupled to a fluid delivery assembly or exchange system 644. For example, the system 644 may include a fluid conduit or tubing 646 extending to an external device, container, or a patient. In some embodiments, the fluid conduit or tubing 646 may extend to a hollow needle or needle assembly, which can be used to inject, extract, or exchange one or more fluids with the integral manifold syringe 600. The fourth port 638 also may be coupled to one or more external devices, containers, or a patient. For example, the fourth port 638 may be coupled to a container of medical fluid, or another integral manifold syringe 600, or a vent system.

The ports 632, 634, 636, and 638 may have a variety of internal and external geometries, connection mechanisms, and positions or arrangements about the stopcock manifold body or housing 628. For example, the ports 632, 634, 636, and 638 may include one or more luer fittings, male threads, female threads, inwardly or outwardly tapered structures, cylindrical structures or tubes, and so forth. For example, the third port 636 may include a threaded luer fitting or connector 648. The fluid flow between these ports 632, 634, 636, and 638 may be controlled before, during, or after a particular medical procedure via the valve lever or manifold flow control actuator 630. As illustrated, the actuator 630 may rotate as indicted by arrow 650.

As discussed in further detail below, the rotational position of the valve lever or manifold flow control actuator 630 may alter or switch the general fluid flow paths between the ports 632, 634, 636, and 638. For example, the actuator 630 may be adjusted to enable injection, extraction, or exchange of a first fluid 652 disposed in the first chamber or cylindrical passage 624 or a second fluid 654 disposed in the second chamber or cylindrical passage 626. The simultaneous or sequential injection, extraction, or general exchange of these first and second fluids 652, 654 may be achieved via movement of the first and second plungers 620, 622 in either inward directions 656 and 658 or outward directions 660, 662, respectively.

The first and second plungers 620, 622 may include first and second proximal plungers or plunger heads 664, 666 coupled to first and second shafts or push rods 668, 670, respectively. The first and second proximal plungers or plunger heads 664, 666 also may include one or more concentric seals, such as o-rings 672, 674. The first and second shafts or push rods 668, 670 also may include first and second rib structures 676, 678 and first and second thumb tabs or peripheral ends 680, 682. Depending on the particular position of the actuator 630, a user may depress or pull the first plunger 620 and/or the second plunger 622 to inject, extract, or generally exchange the first fluid 652 and/or the second fluid 654 between the first and second chambers or cylindrical passages 624, 626, one or more devices coupled to the ports 636, 638, or one or more patients coupled to the ports 636, 638, or a combination thereof.

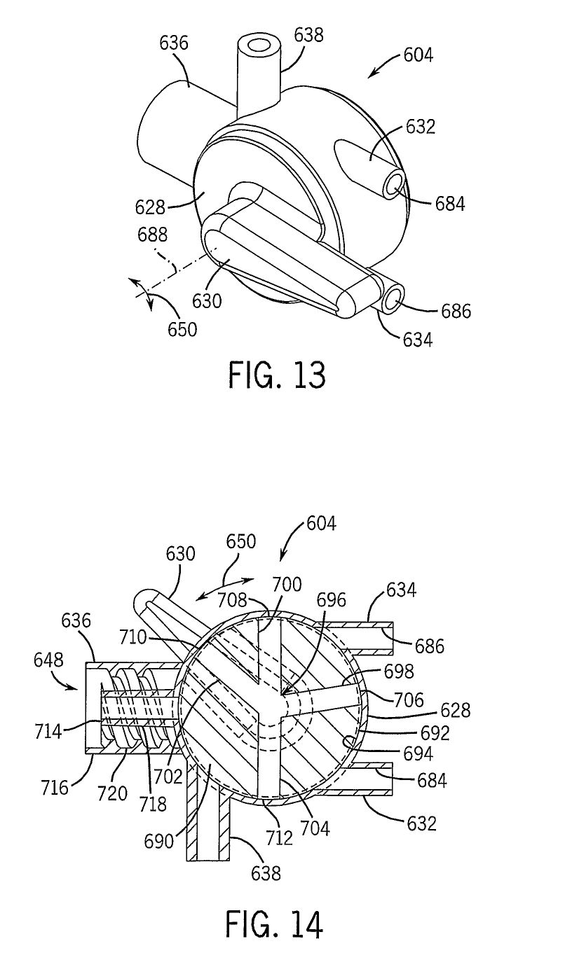

FIG. 13 is a perspective view of an embodiment of the integral stopcock manifold or valve-controlled manifold 604 separate from the multi-barrel syringe 602 for purposes of illustration and discussion. As illustrated, the first, second, and fourth ports 632, 634, and 638 may have generally cylindrical interiors and exteriors to facilitate fixed or removable connections with the first and second syringes 606, 608 and an external device, container, or tubing. For example, a variety of adhesives or bonding materials, such as epoxy, may be disposed within first and second cylindrical interiors 684, 686 of the first and second ports 632, 634, such that the first and second tip fittings or conduits 640, 642 of the unitized multi-barrel body 610 may be fixably or permanently secured to the first and second ports 632, 634 of the integral stopcock manifold or valve-controlled manifold 604. Again, as discussed above, these first and second ports 632, 634 may be coupled to the first and second tip fittings or conduits 640, 642 via one or more mechanical fasteners, bonding materials or adhesives, chemical bonds, welding, or other forms of heat treatment, or combinations thereof. For example, the first and second ports 632, 634 may be laser welded to the first and second tip fittings or conduits 640, 642. Alternatively, the first and second barrels or cylindrical enclosures 612, 614, the intermediate lengthwise joint 616, the flange 618, and the stopcock manifold body or housing 628 may be assembled together and heat treated in an oven or other heat treating device, thereby creating bonds between all of the components to form the integral manifold syringe 600. In this procedure, the first and second plunger 620, 622 along with the actuator 630 and moveable internal components of the integral stopcock manifold or valve-controlled manifold 604 may be installed or assembled after the heat treating procedure. Again, the components of the integral manifold syringe 600 may be secured to one another by a variety of techniques that may reduce the number of potential contamination points between the first and second chambers or cylindrical passages 624, 626 and the patient or an external device. In the illustrated embodiment, the stopcock manifold body or housing 628 may have a generally cup-shaped, barrel-shaped, or generally cylindrical structure, such that the valve lever or manifold flow control actuator 630 may rotate about an axis 688 as indicated by the arrow 650.

FIGS. 14-18 are cross-sectional views of the exemplary integral stopcock manifold or valve-controlled manifold 604 as illustrated in FIG. 13, further illustrating a variety of flow configurations between the various ports 632, 634, 636, and 638. As illustrated in these cross-sectional views, the integral stopcock manifold or valve-controlled manifold 604 may include a rotatable core or manifold interior 690 disposed rotatably inside a stopcock manifold body or housing 628. Specifically, the rotatable core or manifold interior 690 may include a generally solid structure having a cylindrical exterior 692, which may moveable or rotatably interface with a cylindrical interior 694 of the stopcock manifold body or housing 628. The rotatable core or manifold interior 690 also may include a variety of fluid flow passages or pathways that may be selectively aligned with zero, one, two, three, or all of the ports 632, 634, 636, and 638. For example, the illustrated rotatable core or manifold interior 690 may include a multi-passage or pathway arrangement 696, which may include a plurality of branch pathways or passages 698, 700, 702, and 704. However, the multi-passage or pathway arrangement 696 may have different numbers, directions, positions, or general geometries of branch pathways or passages 698 depending on the number and arrangement of ports (e.g., 632, 634, 636, and 638) and the desired number of interconnections between these ports. As illustrated, the branch pathways or passages 698 and 702 are generally in different directions than branch pathways or passages 700 and 704. In fact, the branch pathways or passages 698 and 702 may be described as converging toward or diverging away from the branch pathways or passages 700 and 704. In certain embodiments, the multi-passage or pathway arrangement 696 may include branch pathways or passages to enable each possible interconnection between two, three, or all of the ports as well as a completely disconnected configuration between the various ports. Thus, the rotatable core 690 may be described as a flow control core, a rotatable multi-passage core, a multi-way flow selection mechanism, or a multi-way valve core (e.g., a four-way valve core).

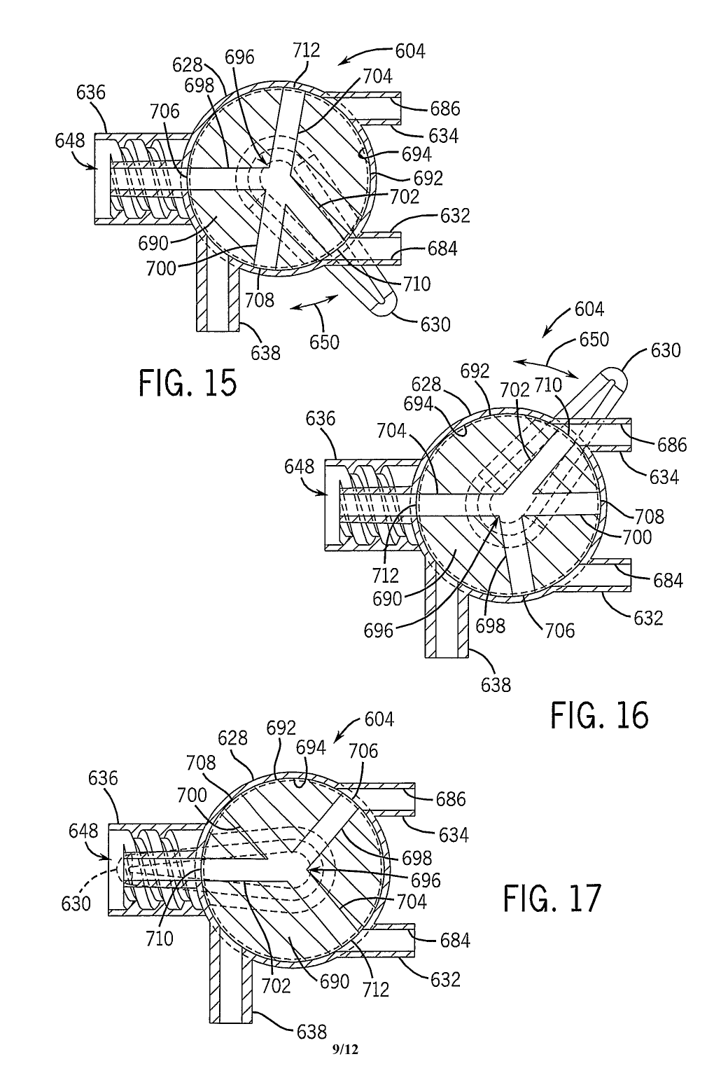

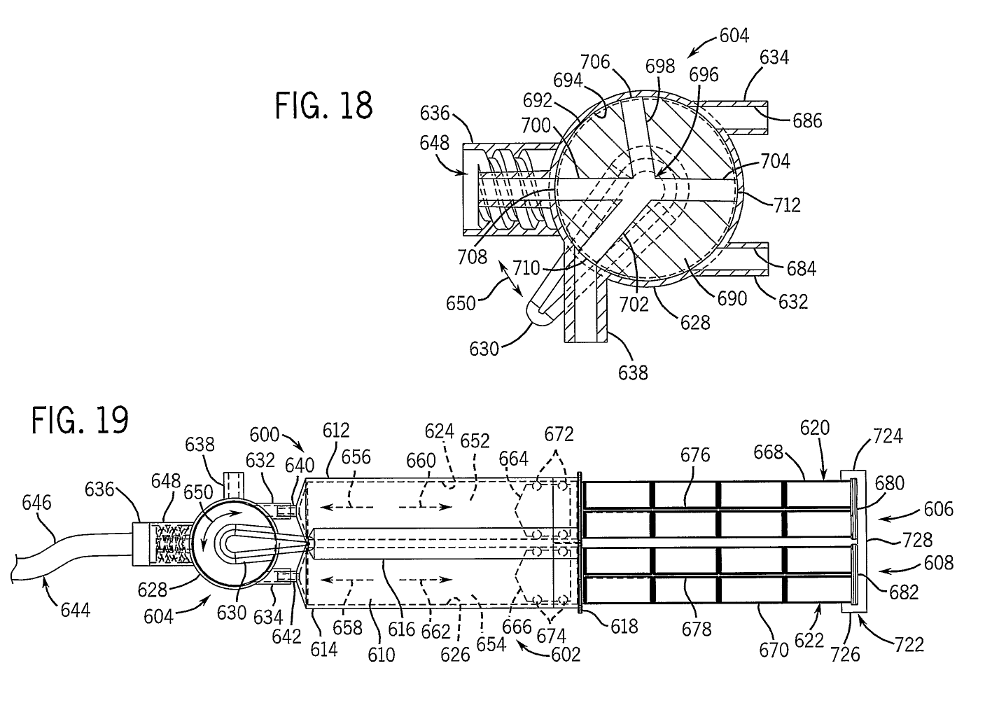

As noted above, the general layout of the branch pathways or passages 698, 700, 702, and 704 may be directly related to the layout of the ports 632, 634, 636, and 638. For example, the branch pathways or passages 698 and 702 may be obtusely angled with respect to one another to generally match with an obtuse angle between the ports 636 and 632, as illustrated and discussed below with reference to FIG. 15. Similarly, the branch pathways or passages 702 and 704 may be obtusely angled with respect to one another to generally match with an obtuse angle between the ports 634 and 636, as illustrated and discussed below with reference to FIG. 16. In addition, the branch pathways or passages 698 and 704 may be acutely, obtusely, or perpendicularly angled with respect to one another to generally match with an acute, obtuse, or perpendicular angle between the ports 634 and 632, as illustrated and discussed below with reference to FIG. 17. Simultaneously or independently, the branch pathway or passage 702 may be acutely angled with respect to one or both of the branch pathways or passages 698 and 704 to generally match with an obtuse angle between the port 636 and one or both of the ports 634 and 632, as illustrated and discussed below with reference to FIG. 17. Referring to FIG. 18, the branch pathways or passages 700 and 702 may be acutely angled with respect to one another to generally match with an acute angle between the ports 636 and 638. Again, the specific angles may vary depending on the desired layout of the ports 632, 634, 636, and 638. The illustrates branch pathways or passages 698, 700, 702, and 704 may include one or more Y-shaped pathways, W-shaped pathways, U-shaped pathways, straight pathways, and so forth.

Turning now to FIG. 14, the valve lever or manifold flow control actuator 630 may be rotated to position the multi-passage or pathway arrangement 696 in a no flow configuration between the various ports 632, 634, 636, and 638. Specifically, the rotatable core or manifold interior 690 may be directly rotated by the valve lever or manifold flow controlled actuator 630, such that the branch pathways or passages 698, 700, 702, and 704 may be disposed intermediate the various ports 632, 634, 636, and 638. In other words, the outer ends or openings 706, 708, 710, and 712 of the branch pathways or passages 698, 700, 702, and 704 may be cut off, closed, or generally sealed against the cylindrical interior 694 of the stopcock manifold body or housing 628 in the illustrated no flow configuration. As further illustrated in FIG. 14, the threaded luer 648 of the third port 636 may include a male luer 714 disposed concentrically within a luer collar 716. The illustrated male luer 714 may have a generally tapered or conical exterior surface 718, such that the male luer 714 may be compressively or wedgingly mated with a female luer. In addition, the luer collar 716 may include internal threads 720 to supplement the male to female luer fitting. For example, the internal threads 720 of the luer collar 716 may rotatingly receive opposite tabs or a spiral arrangement of one or more threads disposed on the exterior of the female luer. Again, each of the ports 632, 634, 636, and 638 may include a variety of geometries and coupling mechanisms, such as luer fittings.

FIG. 15 is a cross-sectional view of the integral stopcock manifold or valve-controlled manifold 604 as illustrated in FIG. 13, further illustrating a first syringe fluid exchange configuration having the first port 632 coupled to the third port 636. Specifically, the valve lever or manifold flow control actuator 630 may be rotated as indicated by arrow 650, thereby directly or simultaneously moving the rotatable core or manifold interior 690 along with the multi-passage or pathway arrangement 696 to position the branch pathways or passages 702, 698 with the ports 632, 636, respectively. In this flow configuration, the first syringe 606 may inject, extract, or exchange the first fluid 652 with a patient, container, device or a combination thereof via the third port 636. However, in the illustrated configuration, the branch pathways or passages 700, 704 may be directed toward the cylindrical interior 694 of the stopcock manifold body or housing 628, such that the openings 708, 712 may be cut off, blocked, closed, or generally sealed by the cylindrical interior 694. Accordingly, only the branch pathways or passages 698, 702 permit fluid exchange, while the branch pathways or passages 700, 704 are not operational in the illustrated configuration of FIG. 15. Moreover, the ports 634, 638 are not fluidly coupled to any of the branch pathways or passages in this configuration of the multi-passage or pathway arrangement 696.

FIG. 16 is a cross-sectional view of the integral stopcock or manifold or valve-controlled manifold 604 as illustrated in FIG. 13, further illustrating a second syringe fluid exchange configuration having the branch pathways or passages 702, 704 extending between the second port 634 and the third port 636. In this configuration, the second syringe 608 may inject, extract, or generally exchange the second fluid 654 with a patient, a device, a container, tubing, or other external target via the multi- passage or pathway arrangement 696. However, in this configuration, the ends or openings 706, 708 of the branch pathways or passages 698, 700 may be cut off, blocked, closed, or generally sealed by the cylindrical interior 694 of the stopcock manifold body or housing 628. In other words, the branch pathways or passages 698, 700 may be inoperable in the illustrated configuration. Furthermore, the first and fourth ports 632, 638 may be cut off or generally sealed from the multi-passage or pathway arrangement 696 and the other ports 634, 636 via the rotatable core or manifold interior 690. In the illustrated configuration, fluid may pass through the multi-passage or pathway arrangement 696 only between the second and third ports 634, 636 without any introduction or removal of fluids via the ports 632, 638.

FIG. 17 is a cross-sectional view of the integral stopcock manifold or valve-controlled manifold 604 as illustrated in FIG. 13, further illustrating a dual syringe fluid exchange configuration having both the first and second ports 632, 634 simultaneously coupled to the third port 636 via the branch pathways or passages 698, 702, and 704. In this configuration, the first and second syringes 606, 608 may be simultaneously or sequentially engaged to inject, extract, or generally exchange the Page 21 of 29 WO 2006/124634 PCT/US2006/018495 first and second fluids 652, 654 with a patient, device, container, or other external target via the one third port 636. However, in the illustrated configuration, the end or opening 708 of the branch pathway or passage 700 may be cut off, closed, or generally sealed by the cylindrical interior 694 of the stopcock manifold body or housing 628. In addition, the fourth port 638 may be generally cut off, blocked, or sealed by the rotatable core or manifold interior 690. As a result, the integral stopcock manifold or valve-controlled manifold 604 may generally disable the injection, extraction, or general exchange of fluid with a patient, device, container, or other object coupled to the fourth port 638.

FIG. 18 is a cross-sectional view of the integral stopcock manifold or valve-controlled manifold 604 as illustrated in FIG. 13, further illustrating a vent configuration or supplemental fluid exchange configuration having the third and fourth ports 636, 638 fluidly coupled together via the multi- passage or pathway arrangement 696. In the illustrated embodiment, the branch pathways or passages 700, 702 may be generally aligned with the third and fourth ports 636, 638, while the other branch pathways or passages 698, 704 may be directed toward the closed cylindrical interior 694. As a result, the ends or openings 706, 712 of the branch pathways or passages 698, 704 may be generally blocked, closed, or sealed by the interface with the cylindrical interior 694 of the stopcock manifold body or housing 628. In this vent configuration, the first and second syringes 606, 608 may be generally disabled due to blockage of the ports 632, 634 by the rotatable core or manifold interior 690. However, the connection of branch pathways or passages 700, 702 with the third and fourth ports 636, 638 may enable air or fluid to vent out through the fourth port 638.

For example, the third port 636 may be coupled to the fluid delivery assembly or exchange system 644 as discussed above with reference to FIG. 12. By further example, the port 636 may be coupled to the fluid conduit or tubing 646, which may lead to a patient. Accordingly, the port 638 may enable blood and/or air to flow or vent outwardly from the fluid delivery assembly or exchange system 644, into the third port 636, through the branch pathways or passages 700, 702, and out through the fourth port 638 to another tubing, container, or target. Alternatively, the fourth port 638 may be coupled to another syringe, such as the integral manifold syringe 600 as illustrated in FIG. 11, or a gravity injection system, or another desirable system or device. In this configuration with addition syringes or injection systems, one or more fluids may be injected into the fourth port 638, through the branch pathways or passages 700, 702, and out through the third port 636. Again, the port 636 may be coupled to the fluid delivery assembly or exchange system 644, such that the fluid may be injected into a patient or directed to another target object, device, or container. In view of the various configurations illustrated with reference to FIGS. 14-18, the integral stopcock manifold or valve-controlled manifold 604 may be adjusted to enable a variety of flow configurations between the various ports 632, 634, 636, and 638 and various syringes, containers, tubing, or patients involved in a medical procedure.

FIG. 19 is a top view of the integral manifold syringe 600 as illustrated in FIGS. 11 and 12, further illustrating a simultaneous multi-syringe injection configuration having a syringe link or coupling 722 extending between the first and second plungers 620, 622. For example, the syringe link or coupling 722 may have a variety of threaded fasteners, non-threaded fasteners, snap fit mechanisms, latches, slots and grooves, or other tool free or quick attachment and release mechanisms. For example, the syringe link or coupling may have a pair of opposite hooks, slots, or channels 724, 726 disposed on opposite ends or sides of an elongated member or integral thumb tab 728. As illustrated, Page 22 of 29 WO 2006/124634 PCT/US2006/018495 the opposite hooks, slots, or channels 724 or 726 may be generally snapped over, hooked around, or generally secured over the first and second thumb tabs or peripheral ends 680, 682 of the first and second plungers 620, 622, respectively. In certain embodiments, the syringe link or coupling 722 may be coupled to the first and second plungers 620, 622 via an adhesive, a bonding material, welding, or various forms of heat treatment. However, a variety of fixed or removable fastening techniques may be employed to connect the syringe link or coupling 722 with the first and second plungers 620, 622. In this configuration, a user may depress the single elongated member or integral thumb tap 728, thereby simultaneously moving both the first and second plungers 620, 622 to inject the first and second fluids 652, 654 through the integral stopcock manifold or valve-controlled manifold 604 and the fluid delivery assembly or exchange system 644.

In certain embodiments, the syringes illustrated and described above with reference to FIGS. 1-19 may be filled or pre-filled with one or more medical fluids, such as contrast agents, radiopharmaceuticals, tagging agents, biocompatible flushes, or combinations thereof. For example, the disclosed multi-barrel syringes, e.g., 20, 21, 100, 200, 300, 400, 500, or 600, may be filled or pre- filled with a first medical fluid in a first barrel and a second medical fluid in a second barrel. The first medical fluid may include a contrast agent for medical imaging, such as magnetic resonance imaging (MRI), computed tomography (CT), radiography (e.g., x-ray), or ultrasound. Alternatively, the first medical fluid may include a radioisotope or radiopharmaceutical for radiation-based treatment or medical imaging, such as positron emission tomography (PET) or single photon emission computed tomography (SPECT). In addition, the second medical fluid may include a biocompatible flush, such as heparin solution, sterilized water, glucose solution, saline, or another suitable substance. The disclosed multi-barrel syringes may be used to inject the first and second medical fluids one after another or simultaneously into a subject or patient. In certain embodiments, the subject may be scanned or generally imaged by a suitable medical diagnostic and/or imaging system, such as listed above. For example, after the contrast agent or radiopharmaceutical enters the blood stream and distributes or focuses on a particular organ or area of interest, the diagnostic and/or imaging system may function to acquire imaging data, process the data, and output one or more images. Thus, the diagnostic and/or imaging system may include detector/acquisition hardware and software, data/image processing hardware and software, data/image storage hardware and software, a display, a printer, a keyboard, a mouse, a computer workstation, a network, and other associated equipment.

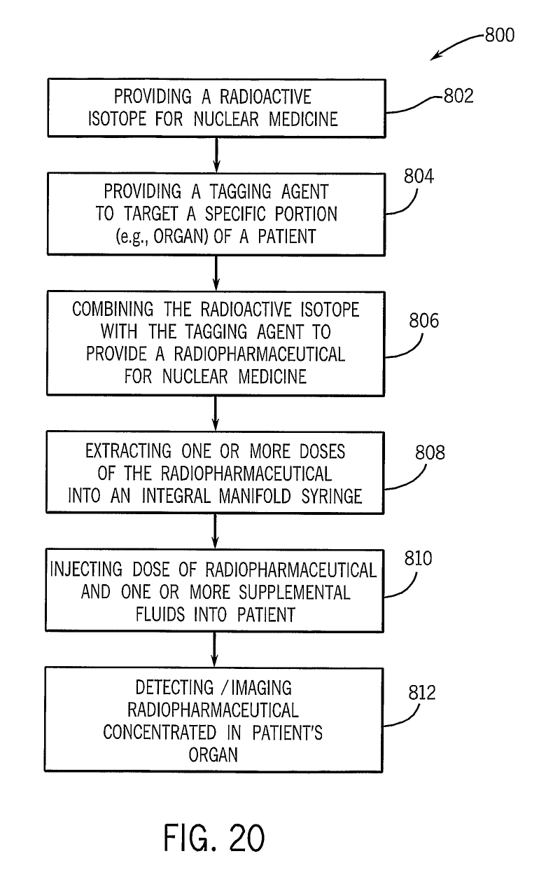

FIG. 20 is a flowchart illustrating an exemplary nuclear medicine process utilizing one or more syringes as illustrated with reference to FIGS. 1-19. As illustrated, the process 800 begins by providing a radioactive isotope for nuclear medicine at block 802. For example, block 802 may include eluting technetium-99m from a radioisotope generator. At block 804, the process 800 proceeds by providing a tagging agent (e.g., an epitope or other appropriate biological directing moiety) adapted to target the radioisotope for a specific portion, e.g., an organ, of a patient. At block 806, the process 800 then proceeds by combining the radioactive isotope with the tagging agent to provide a radiopharmaceutical for nuclear medicine. In certain embodiments, the radioactive isotope may have natural tendencies to concentrate toward a particular organ or tissue and, thus, the radioactive isotope may be characterized as a radiopharmaceutical without adding any supplemental tagging agent. At block 808, the process 800 then may proceed by extracting one or more doses of the Page 23 of 29 WO 2006/124634 PCT/US2006/018495 radiopharmaceutical into a syringe or another container, such as a container suitable for administering the radiopharmaceutical to a patient in a nuclear medicine facility or hospital. In certain embodiments, block 808 includes filling the integral manifold syringe 600 as illustrated in FIGS. 11-19 with a radiopharmaceutical and a flushing solution in the respective first and second syringes 606, 608. At block 810, the process 800 proceeds by injecting or generally administering a dose of the radiopharmaceutical and one or more supplemental fluids into a patient. After a pre-selected time, the process 800 proceeds by detecting/imaging the radiopharmaceutical tagged to the patient's organ or tissue (block 812). For example, block 812 may include using a gamma camera or other radiographic imaging device to detect the radiopharmaceutical disposed on or in or bound to tissue of a brain, a heart, a liver, a tumor, a cancerous tissue, or various other organs or diseased tissue.

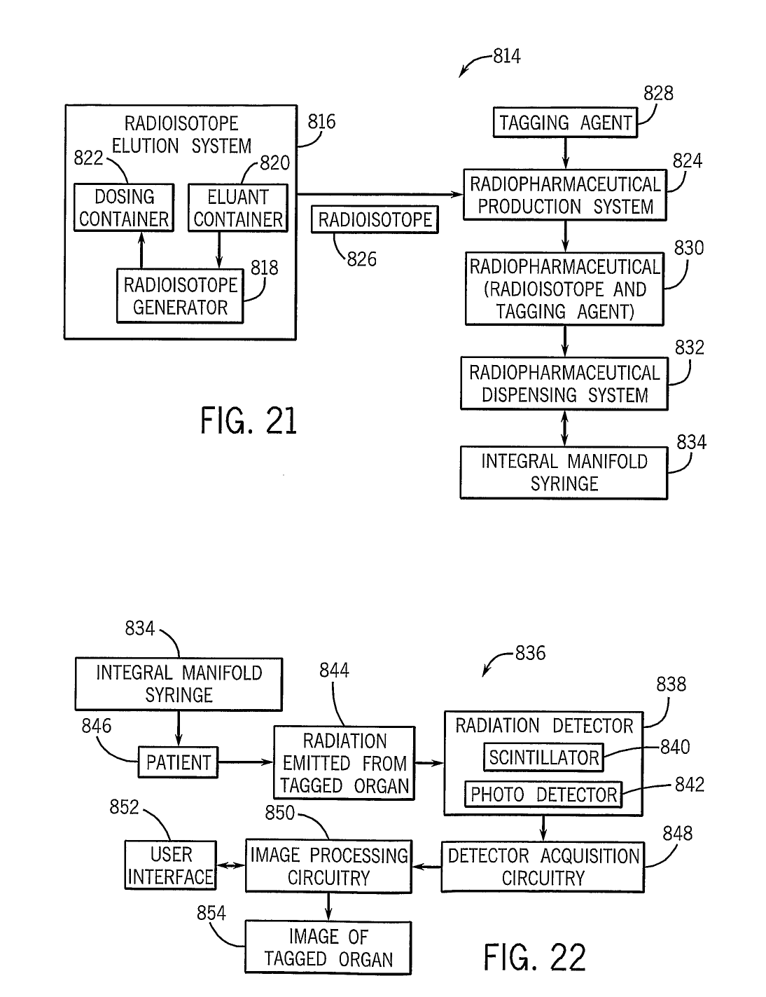

FIG. 21 is a block diagram of an exemplary system 814 for providing a syringe having a radiopharmaceutical disposed therein for use in a nuclear medicine application. For example, the syringe may be one of the syringes illustrated and described with references to FIGS. 1-19. As illustrated, the system 814 may include a radioisotope elution system 816 having a radioisotope generator 818, an eluant supply container 820, and an eluate output container or dosing container 822. In certain embodiments, the eluate output container 822 may be in vacuum, such that the pressure differential between the eluant supply container 820 and the eluate output container 822 facilitates circulation of an eluant (e.g., saline) through the radioisotope generator 818 and out through an eluate conduit into the eluate output container 822. As the eluant, e.g., a saline solution, circulates through the radioisotope generator 818, the circulating eluant generally washes out or elutes a radioisotope, e.g., Technetium-99m. For example, one embodiment of the radioisotope generator 818 may include a radiation shielded outer casing (e.g., lead shell) that encloses a radioactive parent, such as molybdenum-99, adsorbed to the surfaces of beads of alumina or a resin exchange column. Inside the radioisotope generator 818, the parent molybdenum-99 transforms, with a half-life of about 67 hours, into metastable technetium-99m. The daughter radioisotope, e.g., technetium-99m, is generally held less tightly than the parent radioisotope, e.g., molybdenum-99, within the radioisotope generator 818. Accordingly, the daughter radioisotope, e.g., technetium-99m, can be extracted or washed out with a suitable eluant, such as an oxidant-free physiologic saline solution. The eluate output from the radioisotope generator 818 into the eluate output container 822 generally includes the eluant and the washed out or eluted radioisotope from within the radioisotope generator 818. Upon receiving the desired amount of eluate within the eluate container 822, a valve may be closed to stop the eluant circulation and output of eluate. As discussed in further detail below, the extracted daughter radioisotope can then, if desired, be combined with a tagging agent to facilitate diagnosis or treatment of a patient (e.g., in a nuclear medicine facility).

As further illustrated in FIG. 21, the system 814 also may include a radiopharmaceutical production system 824, which functions to combine a radioisotope 826 (e.g., technetium-99m solution acquired through use of the radioisotope elution system 816) with a tagging agent 828. In some embodiments, this radiopharmaceutical production system 824 may refer to or include what are known in the art as “kits” (e.g., Technescan® kit for preparation of a diagnostic radiopharmaceutical). Again, the tagging agent may include a variety of substances that are attracted to or targeted for a particular portion (e.g., organ, tissue, tumor, cancer, etc.) of the patient. As a result, the radiopharmaceutical Page 24 of 29 WO 2006/124634 PCT/US2006/018495 production system 824 produces or may be utilized to produce a radiopharmaceutical including the radioisotope 826 and the tagging agent 828, as indicated by block 830. The illustrated system 814 may also include a radiopharmaceutical dispensing system 832, which facilitates extraction of the radiopharmaceutical into a vial or syringe 834 as illustrated in FIGS. 1-19. In certain embodiments, the various components and functions of the system 814 may be disposed within a radiopharmacy, which prepares the syringe 834 of the radiopharmaceutical for use in a nuclear medicine application. For example, the syringe 834 may be prepared and delivered to a medical facility for use in diagnosis or treatment of a patient. Again, the syringe 834 may be an integral manifold syringe as illustrated and described with reference to FIGS. 11 -19.

FIG. 22 is a block diagram of an exemplary nuclear medicine imaging system 836 utilizing the syringe 834 of radiopharmaceutical provided using the system 814 of FIG. 21. As illustrated, the nuclear medicine imagining system 836 may include a radiation detector 838 having a scintillator 840 and a photo detector 842. In response to radiation 844 emitted from a tagged organ within a patient 846, the scintillator 840 emits light that may be sensed and converted to electronic signals by the photo detector 842. Although not illustrated, the imaging system 836 also can include a collimator to collimate the radiation 844 directed toward the radiation detector 838. The illustrated imaging system 836 also may include detector acquisition circuitry 848 and image processing circuitry 850. The detector acquisition circuitry 848 generally controls the acquisition of electronic signals from the radiation detector 838. The image processing circuitry 850 may be employed to process the electronic signals, execute examination protocols, and so forth. The illustrated imaging system 836 also may include a user interface 852 to facilitate user interaction with the image processing circuitry 850 and other components of the imaging system 836. As a result, the imaging system 836 produces an image 854 of the tagged organ within the patient 846. Again, the foregoing procedures and resulting image 854 directly benefit from the syringe as illustrated and described with reference to FIGS. 1-19.

When introducing elements of various embodiments of the present invention, the articles “a”, “an”, “the”, and “said” are intended to mean that there are one or more of the elements. The terms “comprising”, “including”, and “having” are intended to be inclusive and mean that there may be additional elements other than the listed elements. Moreover, the use of “top”, “bottom”, “above”, “below” and variations of these terms is made for convenience, but does not require any particular orientation of the components.

While the invention may be susceptible to various modifications and alternative forms, specific embodiments have been shown by way of example in the figures and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1-32. (canceled)

33. An integral multi-barrel syringe, comprising:
a first plunger disposed in a first barrel;
a second plunger disposed in a second barrel;
a multi-passage structure coupled to the first and second barrels, wherein the multi-passage structure comprises first and second ports downstream of the first and second barrels;
a one-way valve disposed in the multi-passage structure; and
a multi-way valve disposed in the multi-passage structure, wherein the multi-way valve comprises an actuator.

34. The syringe of claim 33, wherein the multi-way valve is disposed between the first and second ports.

35. The syringe of claim 33, wherein the multi-way valve is disposed at a pathway intersection between the first and second barrels and the first and second ports.

36. The syringe of claim 35, wherein the pathway intersection comprises a three-way intersection between a first passage coupled to the first port, a second passage coupled to the second port, and a third passage coupled to the first and second barrels.

37. The syringe of claim 33, wherein the one-way valve is disposed between the first and second ports.

38. The syringe of claim 37, comprising another one-way valve disposed between the first and second barrels.

39. The syringe of claim 38, wherein the multi-way valve is disposed between the first and second ports.

40. The syringe of claim 33, wherein the one-way valve is disposed between the first and second barrels and the multi-way valve is disposed between the first and second ports.

41. The syringe of claim 33, wherein the multi-way valve comprises a two-way valve.

42. The syringe of claim 33, wherein the multi-way valve comprises a three-way valve.

43. The syringe of claim 33, wherein the multi-way valve comprises a four-way valve.

44. The syringe of claim 33, comprising a fluid delivery mechanism coupled to the first port and a container coupled to the second port.

45. The syringe of claim 33, wherein the second port comprises a vent.

46. The syringe of claim 33, comprising a radiopharmaceutical, a contrast agent, a medical fluid, or a combination thereof disposed in the integral multi-barrel syringe.

47. A method of imaging a patent, the method comprising:
injecting a radiopharmaceutical or contrast media into a patient, wherein the injecting comprises changing fluid flow pathways in a multi-way manifold between a plurality of barrels of a unitized, multi-barrel syringe and a plurality of ports; and
generating an image of the patient based, at least in part, on the radiopharmaceutical or contrast media in the patient.

48. The method of claim 47, wherein the changing comprises selectively rotating a multi-passage core in a manifold housing of the multi-way manifold.

49. The method of claim 48, wherein the selectively rotating comprises aligning one or more passages of the multi-passage core between a plurality of flow pathway positions relative to the plurality of ports and the plurality of barrels.

50. The method of claim 49, wherein the plurality of flow pathway positions comprises a first position having a no flow pathway arrangement, a second position having a first flow pathway arrangement between a first barrel of the plurality of barrels and a first port of the plurality of ports, a third position having a second flow pathway arrangement between a second barrel of the plurality of barrels and the first port of the plurality of ports, a fourth position having a third flow pathway arrangement between the first and second barrels and the first port, or a fifth position having a fourth flow pathway arrangement between the first port and a second port of the plurality of ports, or a combination thereof.

51. The method of claim 47, wherein the changing comprises substantially restricting flow to one-way flow in a first portion of the multi-way manifold and selectively controlling flow to different multi-way flows in a second portion of the multi-way manifold.

52. The method of claim 47, wherein the multi-way manifold comprises a valve mechanism having at least four ports, including first and second ports coupled to first and second barrels of the plurality of barrels.
